SEQUENCE LISTING (4 sequences)

SEQ ID NO: 1 - DNA, 1472 bp, Human RCE1
  CDS Complement((32)..(1021))
gtcactggtgcgcgccgcgggtcagggcgcaatggcggcgctgggcggggatgggctgcgactgctgtcggtgtcgcggccggagcggccgcccgagtcggcggcgctgggcggcctgggccccgggctgtgctgctgggtgtcagtgttctcctgcctcagcctcgcctgctcctacgtgggcagcctctacgtctggaagagcgaactgcccagggaccatcccgcggtcatcaagcgacgcttcaccagcgtcctggtggtgtccagtctctcacccctgtgcgtgctgctctggagggaactcacaggcatccagccaggcacatccctgctcaccctgatgggcttcaggctggagggcattttcccagcggcgctgctgcccctgttgctgaccatgattcttttcctgggcccactgatgcagctctctatggattgcccttgtgacctggcagatgggctgaaggttgtcctggccccccgctcctgggcccgctgcctcacagacatgcgttggctgcggaaccaagtgatcgccccgctgacagaggagctggtgttccgggcctgtatgctgcccatgttagcaccgtgcatgggcctgggccctgctgtgttcacctgcccgctcttttttggagttgcccattttcaccatattattgagcagctgcgtttccgccagagcagcgtggggaacatcttcttgtctgctgcgttccagttctcctacacagctgtcttcggtgcctacactgctttcctcttcatccgcacaggacacctgattgggccggttctctgccattccttctgcaattacatgggtttcccagctgtttgcgcggccttggagcacccacagaggcggcccctgctggcaggctatgccctgggtgtgggactcttcctgcttctgctccagcccctcacggaccccaagctctacggcagccttcccctttgtgtgcttttggagcgggcaggggactcagaggctcccctgtgctcctgacctatgctcctggatacgctatgaactctcaccggctccccagccctccccaccaaggggtactgcaggggaagggctggctggggtccccgagatctcaggaatttttgtaggggattgaagccagagctagttgcgtcccagggaccaagagaaagaagcagatatccaaagggtgcagccccttttgaaaggggtgtttacgagcagctgtgagtgaggggacaaggggcaggtcccaggagccacacactcccttcctcactttggactgctgcttctcttagctcctctgcctctgaaaagctgctcggggttttttatttataaaacctctccccaccccccaccccccaaacttcctgggttttctcattgtctttttgcatcagtactttgtattgggatattaaagagatttaacttgggtaaaaaaaaaaaaaaaaaaaaa

SEQ ID NO: 2 - protein, 329 aa, Human RCE1
MAALGGDGLRLLSVSRPERPPESAALGGLGPGLCCWVSVFSCLSLACSYVGSLYVWKSELPRDHPAVIKRRFTSVLVVSSLSPLCVLLWRELTGIQPGTSLLTLMGFRLEGIFPAALLPLLLTMILFLGPLMQLSMDCPCDLADGLKVVLAPRSWARCLTDMRWLRNQVIAPLTEELVFRACMLPMLAPCMGLGPAVFTCPLFFGVAHFHHIIEQLRFRQSSVGNIFLSAAFQFSYTAVFGAYTAFLFIRTGHLIGPVLCHSFCNYMGFPAVCAALEHPQRRPLLAGYALGVGLFLLLLQPLTDPKLYGSLPLCVLLERAGDSEAPLCS

SEQ ID NO: 3 - DNA, 1401 bp, Mouse RCE1
  CDS Complement((1)..(990))
atggcggcgctgggcggggacgggctgcgtttactgtcggtatcgcggccagagcggcagcccgagtcagccgcgctgagcagcctgggcccagggctgtgctgctgggtgtctgtgttctcctgcttcagcctcgcctgctcctacgtgggcagcctctacgtgtggaagagcgagctgcccagggaccaccccgctgttatcaagcggcgttccaccagtgtcctggtagtgtccagcttgtcccctctttgcgtgctgctctggagggaactcactggcatccagccaggcacatcactgcttaccttgatgggcttcaggctggagggcattttcccagcagcgctgctcgccctgctgctaactatgatccttttcctgggtccactgatgcagctctctatggattgcccttgtgacctgacagatgggctgaaggttgtcctggcccctcgttcttgggcccgctgcctcacagacatgcgctggctacgaaaccaagttattgcaccgctgacagaggagctggtgttccgggcttgcatgctgcccatgctagcgccgtgcacgggtctgggccctgctgtgttcacctgcccactcttttttggagtcgcccattttcaccacattattgagcagctgcgcttccgccagagcagtgtgggaagtatcttcgtgtctgcagcgttccagttctcctacaccgctgtcttcggtgcttatacagctttcctcttcatccgcacaggacacctgatagggccggttctctgccactctttctgcaactacatgggcttccctgcagtgtgtgcagccctggagcatccacagaagtggccactgctggcaggctatgccctcggtgtgggacttttcctgcttctgcttcaacccctgacagaccccaagctctatggcagccttcctctttgtatgcttttggaaagaacaggggcctcagagaccctactgtgctcctgacgatcactcttttgtgcactccagtgaactctgacgggctctccagctcctccttaccaaggaatactgcaagggagggactggctggggtccccgagatctcaggaatttttgtaggggattgaagccagagctagttgaatcccagggaccaagagaaaggagcagatatccaaagggtgcagcccctctcgaaggggggatgagcagcaactggaggtgaggggacaagggcaaatcctaggagctgtggactgacgcttccttggctcctttgcgtcccccctttccccttgaaaagctgctcggtgggtttatttataaaacccctcctctcaacttcccagggttttctcattgtctttttgcatcaagactttgtattgggatattaaagagatttaacttggg

SEQ ID NO: 4 - protein, 329 aa, Mouse RCE1
MAALGGDGLRLLSVSRPERQPESAALSSLGPGLCCWVSVFSCFSLACSYVGSLYVWKSELPRDHPAVIKRRSTSVLVVSSLSPLCVLLWRELTGIQPGTSLLTLMGFRLEGIFPAALLALLLTMILFLGPLMQLSMDCPCDLTDGLKVVLAPRSWARCLTDMRWLRNQVIAPLTEELVFRACMLPMLAPCTGLGPAVFTCPLFFGVAHFHHIIEQLRFRQSSVGSIFVSAAFQFSYTAVFGAYTAFLFIRTGHLIGPVLCHSFCNYMGFPAVCAALEHPQKWPLLAGYALGVGLFLLLLQPLTDPKLYGSLPLCMLLERTGASETLLCS

(19) United States
(12) Patent Application Publication
(10) Pub. No.: US 2003/0013181 A1
(43) Pub. Date: Jan. 16, 2003

(54) NOVEL NUCLEIC ACIDS AND POLYPEPTIDES RELATED TO A FARNESYL-DIRECTED ENDOPEPTIDASE

(76) Inventors: YUN-JUNG CHOI, UNION CITY, CA (US); ANNE K. NORTH, PLEASANT HILL, CA (US); GEORGE A. MARTIN, BERKELEY, CA (US); GIDEON BOLLAG, HERCULES, CA (US)

Correspondence Address: GREGORY GIOTTA PHD, VICE PRESIDENT AND CHIEF LEGAL COUNSEL, ONYX PHARMACEUTICALS INC, 3031 RESEARCH DRIVE, RICHMOND, CA, 94806

(21) Appl. No.: 09/294,455

(22) Filed: Apr. 19, 1999

Related U.S. Application Data
(60) Provisional application No. 60/086,421, filed on May 22, 1998.

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

The present invention relates to a mammalian farnesyl-directed endopeptidase, especially obtainable from a human or mouse. The polypeptide and corresponding nucleic acid are useful in a variety of ways, such as for diagnostic probes, in assays to identify agents which interfere with the endopeptidase activity and its expression, and for the screening of agents for treating cancer and other pathways in which the polypeptide is involved.

### BACKGROUND OF THE INVENTION

Eukaryotic proteins containing a C-terminal CAAX motif undergo a series of modifications which involve prenylation, proteolysis, and methylation leading to the production of a mature and biologically active polypeptide. Ras is an example of a modified prenylated protein. Farnesyl-directed endopeptidases are one class of enzymes involved in processing the prenylated proteins. Because of their involvement in the ras signaling pathway, farnesyl-directed endopeptidases play a fundamental role in various cell processes, including cell proliferation diseases.

### DESCRIPTION OF THE INVENTION

The present invention relates to all aspects of a farnesyl-directed endopeptidase, especially a mammalian farnesyl-directed endopeptidase, such as human or mouse RCE1. An aspect of the invention is an isolated mammalian RCE1 polypeptide or fragments of it, an isolated nucleic acid coding for a mammalian RCE1 or fragments of it, and derivatives of these polypeptides and nucleic acids. Related polypeptides, e.g., polypeptides which are coded for by nucleic acids obtainable by hybridization to a mammalian RCE1 nucleic acid, are another feature of the invention.

The invention also relates to methods of using such polypeptides, nucleic acids, or derivatives thereof, e.g., in therapeutics, diagnostics, and as research tools, e.g., to identify compounds which modulate a mammalian RCE1. The invention also concerns ligands of RCE1, such as antibodies, nucleic acid aptamers, and substrates.

### BRIEF DESCRIPTION OF THE DRAWINGS

FIG. 1 shows a nucleotide and amino acid sequence of a human RCE1.

FIG. 2 shows a complete nucleotide sequence of mouse RCE1

FIG. 3 shows a complete amino acid sequence of mouse RCE1

FIG. 4 shows a shows a comparison between amino acid sequences of human, mouse, and yeast RCE1. A consensus sequence is shown. Regions of amino acid sequence identity are highlighted.

FIG. 5 shows a comparison between the amino acid sequences of human and mouse RCE 1. Regions of non-sequence identity are highlighted.

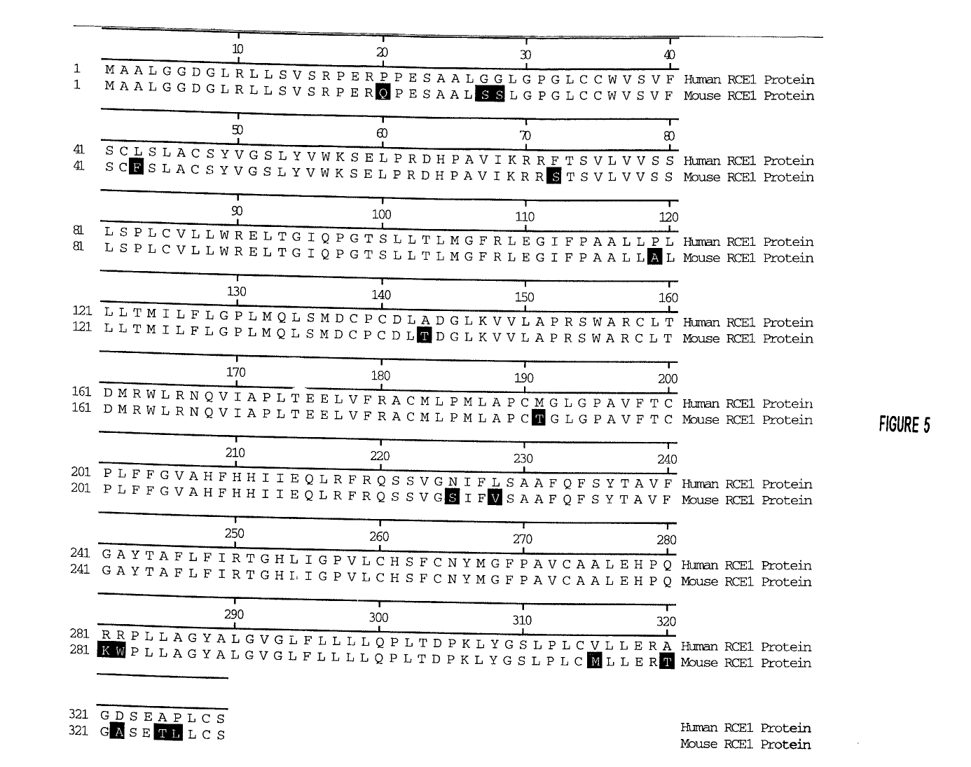

### DETAILED DESCRIPTION OF THE INVENTION

In accordance with the present invention, a novel polypeptide and nucleic acid coding for a mammalian RCE1 polypeptide and nucleic acids have been described. As used herein, an RCE1 polypeptide has an amino acid sequence which is naturally-obtainable and which possesses at least one activity of the following: an endoprotease activity, a substrate binding activity, a transformation-promoting activity, or an RCE1 specific immunogenic activity.

An endoprotease activity of RCE1 means, for example, that the RCE1 is capable of proteolyzing, or enzymatically cleaving, a substrate at an internal amino acid recognition site. Preferably, the endoprotease activity is for a CAAX motif, where A is any aliphatic amino acid and X is any amino acid. In this case, complete cleavage of the substrate results in the production of two fragments, each fragment having a termini defined by the amino acid residue at the cleavage site, e.g., —C—COOH and —NH₂-A. Preferably, the endoprotease activity is dependent upon the attachment of a lipid to the substrate (e.g., at the cysteine residue), such as a cholesterol intermediate, e.g., a 15-carbon farnesyl or 20-carbon geranylgeranyl moiety.

Substrate binding is generally considered the first step in enzyme catalysis because the substrate, acting as a ligand, must first attach to the enzyme surface to enable the enzyme to carry out its catalytic reactions. This enzyme surface can be referred to as the active site of the enzyme. Binding of the substrate to the enzyme surface can involve multiple interactions with the enzyme, e.g., chemical bonding with one or more amino acids and/or functional groups which comprise the enzyme. A substrate binding activity as used herein means that a substrate attaches to the enzyme. Attachment to the enzyme can be accomplished by one or more of the interactions which hold its naturally-occurring substrate to it; however, a polypeptide can have a substrate binding activity when it holds the substrate with less than the naturally-occurring number and quality of interactions. Substrate binding and catalytic activity can be dissociated from each other. Thus, an RCE1 polypeptide in accordance with the invention can possess substrate binding activity but not an endoprotease activity. Substrate binding can optionally be effective: to achieve catalysis of the substrate, to competitively or noncompetitively bind to the active site, to irreversibly attach to the enzyme, to result in the loss of catalytic activity (e.g., where it is a suicide substrate), etc. In a preferred aspect of the invention, the substrate comprises the CAAX motif.

By the term “transformation-promoting activity,” it is meant an activity that produces a transformed phenotype of cells, e.g., induces cell division, induces anchorage independent growth, increases ras activity, etc. The effect can be partial or incomplete. For example, expression of a RCE1 gene in cells can cause a transformed phenotype, or it can enhance the phenotype of already transformed cells.

Immunogenic activity means that the polypeptide is capable of eliciting an immune response specific for an RCE1. The immune response can be a humoral (e.g., induction of antibodies), cellular, or a combination thereof.

The above-mentioned activities of an RCE1 can be assayed, e.g., as described below in the examples or according to methods which the skilled worker would know. For example, endoprotease activity can be measured as described in the examples below. See also, e.g., Methods in Enzymology, 250:251-266, 1995; Boyartchuk et al., Science, 275:1796, 1997. Substrate binding activity can be measured conventionally. For instance, a competition binding assay can be employed to identify substrates which attach to a polypeptide, or derivative thereof, e.g., by combining under effective conditions, a substrate containing a detectable marker, an RCE1 polypeptide, or fragments thereof, and a compound which is to be tested for substrate binding activity. The assay can be accomplished in liquid phase, where bound and free substrate is separated by a membrane, or, it can be accomplished in solid phase, as desired. Solid-phase assays can be performed using high through-put procedures, e.g., on chips, wafers, etc.

A mammalian RCE1 polypeptide is a mammalian polypeptide having an amino acid sequence which is obtainable from a natural source. It therefore includes naturally-occurring, normal, mutant, polymorphic, etc., amino acid sequences which can be obtained from natural populations. Natural sources include, e.g., living cells, e.g., obtained from tissues or whole organisms, cultured cell lines, including primary and immortalized cell lines, biopsied tissues, etc. The present invention also relates to fragments of a full-length mammalian RCE1 polypeptide. The fragments are preferably biologically-active. By biologically-active, it is meant that the polypeptide fragment possesses an activity in a living system or with components of a living system. Biological-activities include those mentioned, e.g., an endoprotease activity, a substrate binding activity, a transformation-promoting activity, and/or an immunogenic activity. Fragments can be prepared according to any desired method, including, chemical synthesis, genetic engineering, cleavage products, etc. See, below.

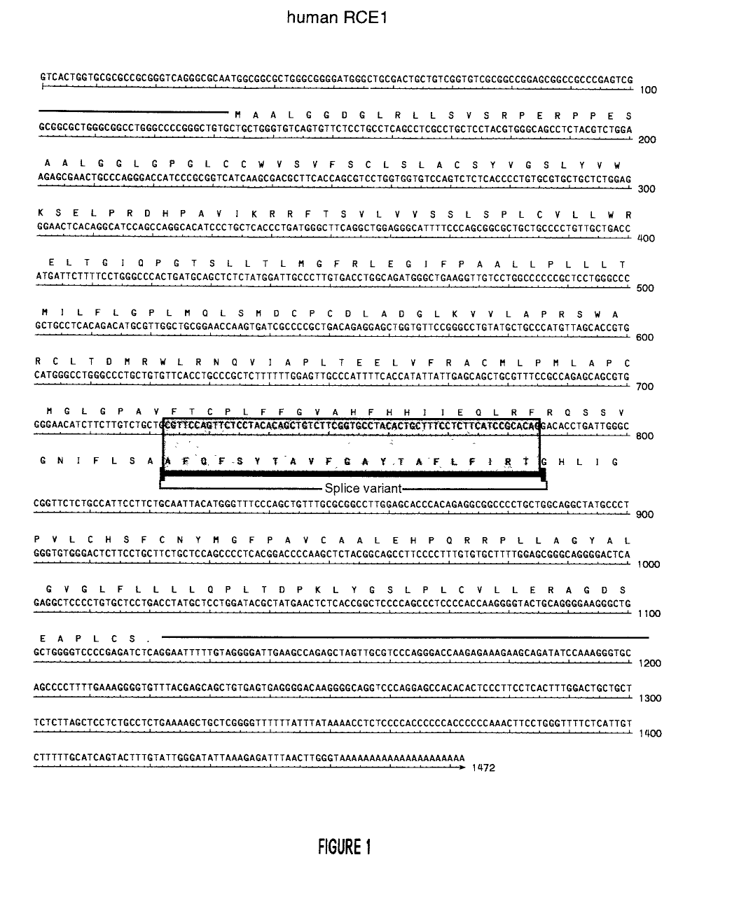

The present invention also relates to a human RCE1 having an amino acid sequence of amino acids 1 to 329; a variant containing contiguously amino acids 1-230 and 252-329; amino acids 231-251; amino acids 19-329. See, FIG. 1. The 329-amino acid polypeptide has a predicted molecular weight of about 35.8 kDa.

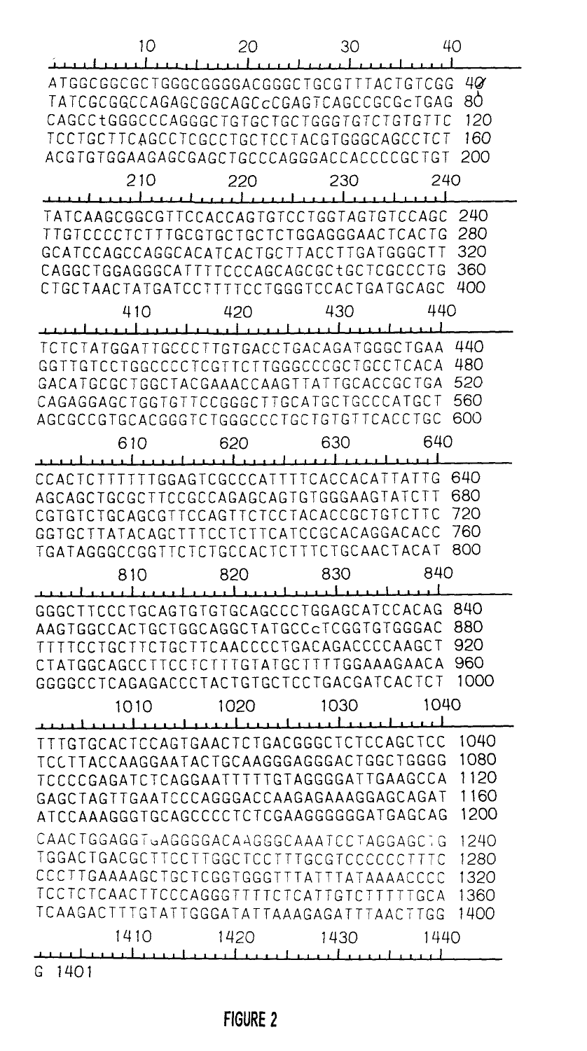

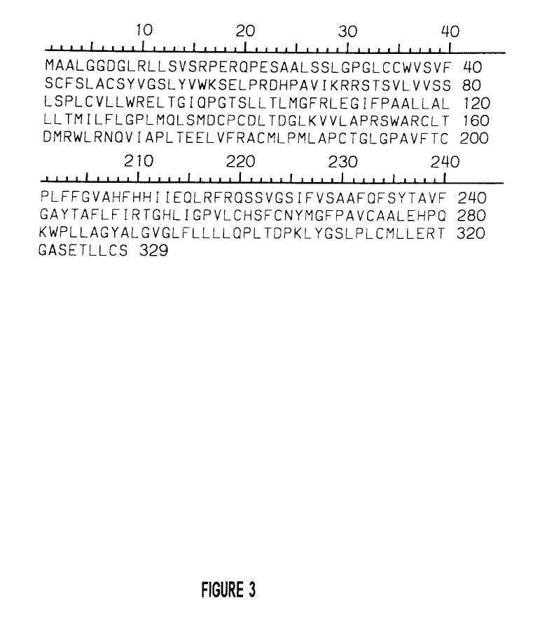

In addition to the human RCE1 sequence, RCE1 sequences from another mammalian species, mouse, has been cloned and identified. These sequences include: AA021859, AA072190, AA154658, AA154864, AA168614, AA218396, AA619282, AA790517, C77052, C86966, W14344, W57162. Thus, the invention relates to a full-length mouse RCE1 sequence as shown in FIG. 2 and FIG. 3.

Other homologs from mammalian and non-mammalian can be obtained according to various methods. For example, hybridization with an oligonucleotide (see below) selective for RCE1 can be employed to select such homologs, e.g., as described in Sambrook et al., Molecular Cloning, 1989, Chapter 11. Such homologs can have varying amounts of nucleotide and amino acid sequence identity and similarity to RCE1. Non-mammalian organisms include, e.g., vertebrates, invertebrates, zebra fish, chicken, Drosophila, C elegans, roundworms, prokaryotes, plants, Arabidopsis, viruses, etc.

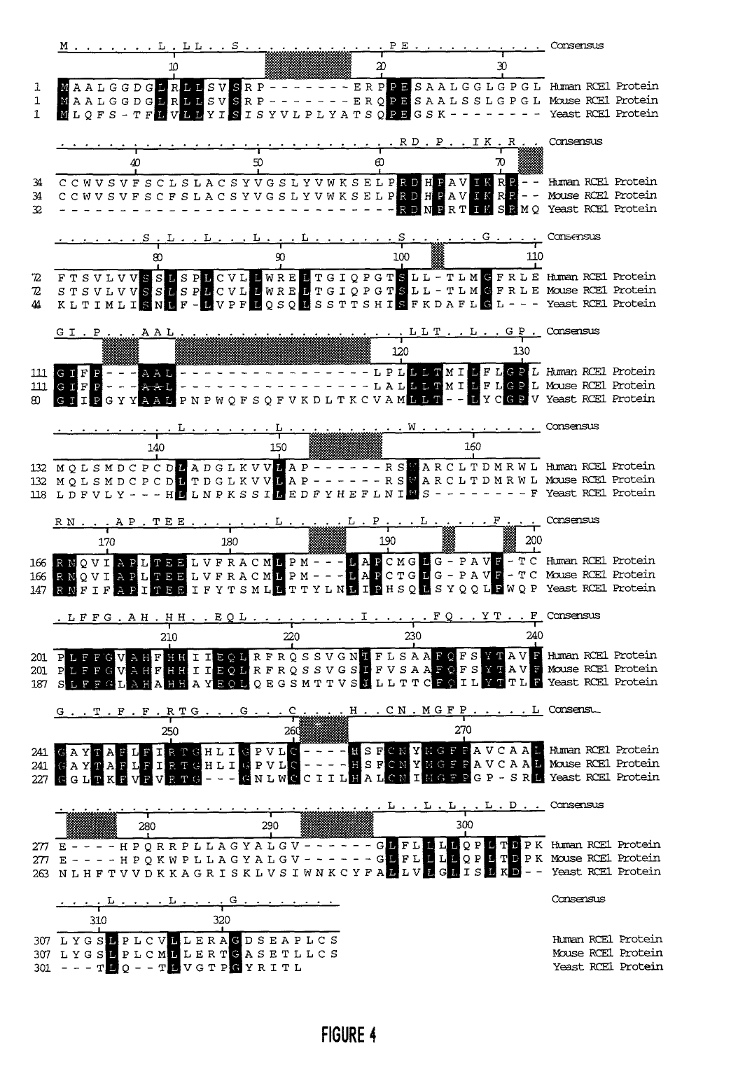

The invention also relates to RCE1 specific or unique amino acid sequences, e.g., a defined amino acid sequence which is found in the particular RCE1 sequence but not in another amino acid sequence. A specific amino acid sequence can be found routinely, e.g., by searching a gene/protein database using the BLAST set of computer programs. Such specific sequences include, e.g., human and mouse but not yeast RCE1; human but not mouse or yeast RCE1; mouse but not human or yeast RCE1. Human and mouse RCE1 specific sequences include e.g., AALGGD, TGIQPGT, MQLSMDCPCD, DGLKVV, ARCLTDMRWL, LVFRACM, RFRQSSVG, and PKLYGS. See FIG. 4.

A mouse or human RCE1 specific or unique amino acid sequence, when possessing an immunogenic activity, can be useful to produce peptides as antigens to generate an immune response specific for RCE. Antibodies obtained by such immunization can be used as a specific probe for RCE protein for diagnostic or research purposes.

A polypeptide of the invention, e.g., having a polypeptide sequence as shown in FIG. 1 and FIG. 3 can by analyzed by available methods to identify structural and/or functional domains in the polypeptide. For example, when the polypeptide coding sequence set forth in FIG. 1 is analyzed by hydropathy and hydrophilicity analysis (e.g., Kyte and Doolittle, J. Mol. Bio.,157:105, 1982) putative membrane spanning regions are identified at A25-W56, F72-W89; L109-M136; A181-F209, V223-1249; T251-L276, and L284-L302. Various other programs can be used to analyze its structure and routinely predict functional domains, including, EMBL Protein Predict; Rost and Sander, Proteins, 19:55-72, 1994.

A polypeptide of the present invention can also have 100% or less amino acid sequence identity to the amino acid sequence set forth in FIG. 1. For the purposes of the following discussion: Sequence identity means that the same nucleotide or amino acid which is found in the sequence set forth in FIG. 1, FIG. 2, or FIG. 4 is found at the corresponding position of the compared sequence(s), e.g., yeast RCE1. See, FIG. 4. A polypeptide having less than 100% sequence identify to the amino acid sequence set forth in FIG. 1 or 3 can contain various substitutions from the naturally-occurring sequence, including homologous amino acid substitutions. See below for examples of homologous amino acid substitution. The sum of the identical and homologous-residues divided by the total number of residues in the sequence over which the RCE1 polypeptide is compared is equal to the percent sequence similarity. For purposes of calculating sequence identity and similarity, the compared sequences can be aligned and calculated according to any desired method, algorithm, computer program, etc., including, e.g., FASTA, BLASTA. A polypeptide having less than 100% amino acid sequence identity to the amino acid sequence of FIG. 1 can comprise e.g., about 99%, 97%, 95%, but greater than 35% identity. A preferred amount of sequence identity is about greater than 94% (e.g., human and mouse exhibit 94% sequence identity).

A RCE1 polypeptide, fragment, or substituted polypeptide can also comprise various modifications, where such modifications include lipid modification such as prenylation (e.g., 15-carbon farnesyl, 20-carbon geranylgeranyl) or other cholesterol intermediates and derivatives, methylation, phosphorylation, glycosylation, covalent modifications (e.g., of an R-group of an amino acid), amino acid substitution, amino acid deletion, or amino acid addition. Modifications to the polypeptide can be accomplished according to various methods, including recombinant, synthetic, chemical, etc.

A mutation to a RCE1 polypeptide can be selected to have a biological activity of RCE1, e.g., an endoprotease activity, a substrate binding activity, a transformation-promoting activity, or an immunogenic activity. The selection and preparation of such mutations is discussed below.

Polypeptides of the present invention (e.g., RCE1, fragments thereof, mutations thereof) can be used in various ways, e.g., in assays, as immunogens for antibodies as described below, as biologically-active agents (e.g., having one or more of the activities associated with RCE1 ). Fragments having ras substrate binding activity, and optionally lacking other biological activities, can be utilized to block ras processing. Such fragments can be administered as DNA (e.g., in vectors, naked DNA, etc.) or they can be administered in forms that can penetrate cells, e.g., in liposomes, conjugated to phagocytosed agents, etc. A useful fragment can be identified routinely by testing the ability of overlapping fragments of the entire length of RCE1 to inhibit an RCE1 activity. The measurement of these activities is described below and in the examples. These peptides can also be identified and prepared as described in EP496 162. Peptides can be chemically-modified, etc.

An RCE1 polypeptide, a derivative thereof, or a fragment thereof, can be combined with one or more structural domains, functional domains, detectable domains, antigenic domains, and/or a desired polypeptides of interest, in an arrangement which does not occur in nature, i.e., not naturally-occurring, e.g., as in an RCE1 gene, a genomic fragment prepared from the genome of a living organism, e.g., an animal, preferably a mammal, such as human, mouse, or cell lines thereof. A polypeptide comprising such features is a chimeric or fusion polypeptide. Such a chimeric polypeptide can be prepared according to various methods, including, chemical, synthetic, quasi-synthetic, and/or recombinant methods. A chimeric nucleic acid coding for a chimeric polypeptide can contain the various domains or desired polypeptides in a continuous or interrupted open reading frame, e.g., containing introns, splice sites, enhancers, etc. The chimeric nucleic acid can be produced according to various methods. See, e.g., U.S. Pat. No. 5,439,819. A domain or desired polypeptide can possess any desired property, including, a biological function such as catalytic, signalling, growth promoting, cellular targeting (e.g., signal sequence, targeting sequence, such as to endosomes, lysosomes, ER, nucleus), etc., a structural function such as hydrophobic, hydrophilic, membrane-spanning, etc., receptor-ligand functions, and/or detectable functions, e.g., combined with enzyme, fluorescent polypeptide, green fluorescent protein, (Chalfie et al., 1994, Science, 263:802; Cheng et al., 1996, Nature Biotechnology, 14:606; Levy et al., 1996, Nature Biotechnology, 14:610, etc. In addition, an RCE1 polypeptide, or a part of it, can be used as selectable marker when introduced into a host cell. For example, a nucleic acid coding for an amino acid sequence according to the present invention can be fused in frame to a desired coding sequence and act as a tag for purification, selection, or marking purposes. The region of fusion can encode a cleavage site to facilitate expression, isolation, purification, etc.

A polypeptide according to the present invention can be produced in an expression system, e.g., in vivo, in vitro, cell-free, recombinant, cell fusion, etc. according to the present invention. Modifications to the polypeptide imparted by such system include, glycosylation, amino acid substitution (e.g., by differing codon usage), polypeptide processing such as digestion, cleavage, endopeptidase or exopeptidase activity, attachment of chemical moieties, including lipids (prenylation), phosphates, etc.

A polypeptide according to the present invention can be recovered from natural sources, transformed host cells (culture medium or cells) according to the usual methods, including, detergent extraction (e.g., CHAPSO, octylglucoside), ammonium sulfate or ethanol precipitation, acid extraction, anion or cation exchange chromatography, phosphocellulose chromatography, hydrophobic interaction chromatography, hydroxyapatite chromatography and lectin chromatography. Protein refolding steps can be used, as necessary, in completing the configuration of the mature protein. Finally, high performance liquid chromatography (HPLC) can be employed for final purification steps.

A mammalian RCE1 nucleic acid, or fragment thereof, is a nucleic acid having a nucleotide sequence obtainable from a natural source, or comprising a naturally-obtainable coding sequence for a mammalian RCE1 polypeptide. See, above. It therefore includes naturally-occurring sequences from normal, mutant, polymorphic, degenerate sequences, etc., alleles which can be obtained from natural populations. Natural sources include, e.g., living cells obtained from tissues and whole organisms, cultured cell lines, including primary and immortalized cell lines. Human RCE1 is expressed in, e.g., heart, brain, placenta, lung, liver, skeletal muscle, kidney, pancrease, spleen, thymus, prostate, testis, ovary, small intestine, colon, and peripheral blood leucocytes. It is also expressed in various cancer cells, including, HL-60, Hela cell S3, chronic myelogenous leukemia K-562, lymphoblastic leukemia MOLT-4, Burkitt's lymphoma Raji, colorectal adenocarcinoma SW 480, lung carcinoma A549, and melanoma G361.

A nucleic acid sequence of a human allele of RCE1 is shown in FIG. 1. contains an open-reading frame of 329 amino acids at nucleotide positions 32 to 1021. A splice variant of such nucleic acid is also illustrated in FIG. 1, containing an open-reading frame of 308 amino acids at nucleotide positions to 32 to 722 and 786-1021. The invention also relates to nucleotides 723 to 785 (useful fragments thereof), absent in the splice variant, which can be used, e.g., as a probe to detect mRNA expression. A nucleic acid sequence of the invention can contain the complete coding sequence from amino acid 1 to amino acid 329, degenerate sequences thereof, and fragments thereof. A nucleic acid according to the present invention can also comprise a nucleotide sequence which is 100% complementary, e.g., an anti-sense, to any nucleotide sequence mentioned above and below.

A nucleic acid according to the present invention can be obtained from a variety of different sources. It can be obtained from DNA or RNA, such as polyadenylated mRNA, e.g., isolated from tissues, cells, or whole organism. The nucleic acid can be obtained directly from DNA or RNA, or from a cDNA library. The nucleic acid can be obtained from a cell at a particular stage of development, having a desired genotype, phenotype (e.g., an oncogenically transformed cell or a cancerous cell), etc.

A nucleic acid comprising a nucleotide sequence coding for a polypeptide according to the present invention can include only a coding sequence of an RCE1; a coding sequence of an RCE1 and additional coding sequence (e.g., sequences coding for leader, secretory, targeting, enzymatic, fluorescent or other diagnostic peptides), coding sequence of RCE1 and non-coding sequences, e.g., untranslated sequences at either a 5′ or 3′ end, or dispersed in the coding sequence, e.g., introns. A nucleic acid comprising a nucleotide sequence coding without interruption for an RCE1 polypeptide means that the nucleotide sequence contains an amino acid coding sequence for an RCE1 polypeptide, with no non-coding nucleotides interrupting or intervening in the coding sequence, e.g., absent intron(s). Such a nucleotide sequence can also be described as contiguous. A genomic DNA coding for an RCE1 can be obtained routinely.

A nucleic acid according to the present invention also can comprise an expression control sequence operably linked to a nucleic acid as described above. The phrase “expression control sequence” means a nucleic acid sequence which regulates expression of a polypeptide coded for by a nucleic acid to which it is operably linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can be heterologous or endogenous to the normal gene.

A nucleic acid in accordance with the present invention can be selected on the basis of nucleic acid hybridization. The ability of two single-stranded nucleic acid preparations to hybridize together is a measure of their nucleotide sequence complementarity, e.g., base-pairing between nucleotides, such as A-T, G-C, etc. The invention thus also relates to nucleic acids which hybridize to a nucleic acid comprising a nucleotide sequence as set forth in FIG. 1 and FIG. 2. A nucleotide sequence hybridizing to the latter sequence will have a complementary nucleic acid strand, or act as a template for one in the presence of a polymerase (i.e., an appropriate nucleic acid synthesizing enzyme). The present invention includes both strands of nucleic acid, e.g., a sense strand and an anti-sense strand.

Hybridization conditions can be chosen to select nucleic acids which have a desired amount of nucleotide complementarity with the nucleotide sequence set forth in FIG. 1 or 2. A nucleic acid capable of hybridizing to such sequence, preferably, possesses 85%, 90%, more preferably 95%, 99%, or more, complementarity, between the sequences. The present invention particularly relates to DNA sequences which hybridize to the nucleotide sequence set forth in FIG. 1 and FIG. 2 under stringent conditions. As used here, stringent conditions means any conditions in which hybridization will occur where there is at least about 85%, about 94%, preferably 97%, nucleotide complementarity between the nucleic acids. Stringent conditions include: 50% formamide, 6×SSC or 6×SSPE, and optionally, a blocking agent (s)s (e.g., Denhardt's reagent; BLOTTO, heparin, denatured, fragmented salmon sperm DNA) at 42 C (or 68° C. if the formamide is omitted). Washing and hybridization can be performed as described in Sambrook et al., Molecular Cloning, 1989, Chapter 9. Hybridization can also be based on calculation of the melting temperature (Tm) of the hybrid formed between the probe and its target, as described in Sambrook et al. Nucleic acids which are preferably excluded are: AA021859, AA072190, AA154658, AA154864, AA168614, AA218396, AA619282, AA790517, C77052, C86966, W14344, W57162, yeast RCE1, or a fragment of yeast RCE1.

According to the present invention, a nucleic acid or polypeptide can comprise one or more differences in the nucleotide or amino acid sequence set forth in FIG. 1, 2, or 3. Changes or modifications to the nucleotide and/or amino acid sequence can be accomplished by any method available, including directed or random mutagenesis.

A nucleic acid coding for an RCE1 according to the invention can comprise nucleotides which occur in a naturally-occurring RCE1 gene e.g., naturally-occurring: polymorphisms, normal or mutant alleles (nucleotide or amino acid), mutations which are discovered in a natural population of mammals, such as humans, monkeys, pigs, mice, rats, or rabbits. By the term naturally-occurring, it is meant that the nucleic acid is obtainable from a natural source, e.g., animal tissue and cells, body fluids, tissue culture cells, forensic samples. Naturally-occurring mutations to RCE1 can include deletions (e.g., a truncated amino- or carboxy-terminus), substitutions, or additions of nucleotide sequence. These genes can be detected and isolated by nucleic acid hybridization according to methods which one skilled in the art would know. It is recognized that, in analogy to other oncogenes, naturally-occurring variants of RCE1 include deletions, substitutions, and additions which produce pathological conditions in the host cell and organism.

A nucleotide sequence coding for a RCE1 polypeptide of the invention can contain codons found in a naturally-occurring gene, transcript, or cDNA, for example, e.g., as set forth in FIG. 1, 2, or 3, or it can contain degenerate codons coding for the same amino acid sequences.

Modifications to an RCE1 sequence, e.g., mutations, can also be prepared based on homology searching from gene data banks, e.g., Genbank, EMBL. Sequence homology searching can be accomplished using various methods, including algorithms described in the BLAST family of computer programs, the Smith-Waterman algorithm, etc. For example, homologous amino acids can be identified between various sequences, such as the human and yeast RCE1 and used as the basis to make amino acid substitutions. See, e.g., FIG. 2.

A mutation(s) can then be introduced into an RCE1 sequence by identifying and aligning amino acids conserved between the polypeptides and then modifying an amino acid in a conserved or non-conserved position.

A nucleic acid and corresponding polypeptide of the present invention include sequences which differ from the nucleotide sequence of FIG. 1 or FIG. 2 but which are phenotypically silent. These sequence modifications include, e.g., nucleotide substitution which do not affect the amino acid sequence (e.g., different codons for the same amino acid or degenerate sequences), replacing naturally-occurring amino acids with homologous amino acids, e.g., (based on the size of the side chain and degree of polarization) small nonpolar: cysteine, proline, alanine, threonine; small polar:serine, glycine, aspartate, asparagine; large polar: glutamate, glutamine, lysine, arginine; intermediate polarity: tyrosine, histidine, tryptophan; large nonpolar: phenylalanine, methionine, leucine, isoleucine, valine.

Homologous acids can also be grouped as follows: uncharged polar R groups, glycine, serine, threonine, cysteine, tyrosine, asparagine, glutamine; acidic amino acids (negatively charged), aspartic acid and glutamic acid; basic amino acids (positively charged), lysine, arginine, histidine.

Homologous amino acids also include those described by Dayhoff in the Atlas of Protein Sequence and Structure 5 (1978), and by Argos in EMBO J., 8, 779-785(1989).

A nucleic acid can comprise a nucleotide sequence coding for a polypeptide having an amino acid sequence as set forth in FIG. 1 or FIG. 3, except where one or more positions are substituted by conservative amino acids; or a nucleotide sequence coding for a polypeptide having an amino acid sequence as set forth in FIG. 1 or 3, except having 1, 5, 10, 15, or 20 substitutions, e.g., wherein the substitutions are conservative amino acids. The invention also relates to polypeptides coded for by such nucleic acids. In addition, it may be desired to change the codons in the sequence to optimize the sequence for expression in a desired host.

A nucleic acid according to the present invention can comprise, e.g., DNA, RNA, synthetic nucleic acid, peptide nucleic acid, modified nucleotides, or mixtures. A DNA can be double- or single-stranded. Nucleotides comprising a nucleic acid can be joined via various known linkages, e.g., ester, sulfamate, sulfamide, phosphorothioate, phosphoramidate, methylphosphonate, carbamate, etc., depending on the desired purpose, e.g., resistance to nucleases, such as RNase H, improved in vivo stability, etc. See, e.g., U.S. Pat. No. 5,378,825.

Various modifications can be made to the nucleic acids, such as attaching detectable markers (avidin, biotin, radioactive elements), moieties which improve hybridization, detection, or stability. The nucleic acids can also be attached to solid supports, e.g., nitrocellulose, magnetic or paramagnetic microspheres (e.g., as described in U.S. Pat. Nos. 5,411,863; U.S. Pat. No. 5,543,289; e.g., comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, 5,478,893.

Another aspect of the present invention relates to oligonucleotides and nucleic acid probes. Such oligonucleotides or nucleic acid probes can be used, e.g., to detect, quantitate, or isolate a RCE1 nucleic acid in a test sample. Detection can be desirable for a variety of different purposes, including research, diagnostic, and forensic. For diagnostic purposes, it may be desirable to identify the presence or quantity of a RCE1 nucleic acid sequence in a sample, where the sample is obtained from tissue, cells, body fluids, etc. In a preferred method, the present invention relates to a method of detecting a RCE1 nucleic acid comprising, contacting a target nucleic acid in a test sample with an oligonucleotide under conditions effective to achieve hybridization between the target and oligonucleotide; and detecting hybridization. An oligonucleotide in accordance with the invention can also be used in synthetic nucleic acid amplification such as PCR (e.g., Saiki et al., 1988, Science, 241:53; U.S. Pat. No. 4,683,202; PCR Protocols: A Guide to Methods and Applications, Innis et al., eds., Academic Press, New York, 1990) or differential display (See, e.g., Liang et al., Nucl. Acid. Res., 21:3269-3275, 1993; U.S. Pat. No. 5,599,672; WO97/18454). Useful oligonucleotides include, e.g.,nucleotides 723-785 of FIG. 1;

5° CAGTGTTCTCCTGCCTCAGCCT 3′ (sense);

5′ TCCATAGAGAGCTGCATCAGTG 3′ (antisense);

5′ CCTCACAGACATGCGTTGGCTGCGGAAC 3′ (sense); and

5′ GGGTGCTCCAAGGCCGCGCAAAC3′ (antisense).

Detection can be accomplished in combination with oligonucleotides for other genes, such as ras. For methods and probes, e.g., U.S. Pat. No. 5,591,582.

Another aspect of the present invention is a nucleotide sequence which is unique to RCE1. By a unique sequence to RCE1, it is meant a defined order of nucleotides which occurs in RCE1, e.g., in the nucleotide sequence of FIG. 1 or FIG. 2, but rarely or infrequently in other nucleic acids, especially not in an animal nucleic acid, preferably mammal, such as human, rat, mouse, etc. Both sense and antisense nucleotide sequences are included. A unique nucleic acid according to the present invention can be determined routinely. A nucleic acid comprising a unique sequence of RCE1 can be used as a hybridization probe to identify the presence of RCE1 in a sample comprising a mixture of nucleic acids, e.g., on a Northern blot. Hybridization can be performed under stringent conditions to select nucleic acids having at least 95% identity (i.e., complementarity) to the probe, but less stringent conditions can also be used. A unique RCE1 nucleotide sequence can also be fused in-frame, at either its 5′ or 3′ end, to various nucleotide sequences as mentioned throughout the patent, including coding sequences for other parts of RCE1, enzymes, GFP, etc., expression control sequences, etc.

Hybridization can be performed under different conditions, depending on the desired selectivity, e.g., as described in Sambrook et al., Molecular Cloning, 1989. For example, to specifically detect RCE1, an oligonucleotide can be hybridized to a target nucleic acid under conditions in which the oligonucleotide only hybridizes to RCE1, e.g., where the oligonucleotide is 100% complementary to the target. Different conditions can be used if it is desired to select target nucleic acids which have less than 100% nucleotide complementarity, at least about, e.g., 99%, 97%, 95%, 90%, 70%, 67%. Since a mutation in a RCE1 can cause diseases or pathological conditions, e.g., cancer, benign tumors, an oligonucleotide according to the present invention can be used diagnostically. For example, a patient having symptoms of a cancer or other condition associated with the Ras signaling pathway (see below) can be diagnosed with the disease by using an oligonucleotide according to the present invention, in polymerase chain reaction followed by DNA sequencing to identify whether the sequence is normal, in combination with other oligonucleotides to oncogenes or genes in the ras signalling pathway, etc., e.g., GRB2, H-, K- and N-ras, c-Raf, MAP kinases, p42, p44, Ser/Thr kinases, Elk-1, c-myc, c-Jun, G-proteins, Ftase, PPSEP, PPSMT, etc. In a preferred method, the present invention relates to a method of diagnosing a cancer comprising contacting a sample comprising a target nucleic acid with an oligonucleotide under conditions effective to permit hybridization between the target and oligonucleotide; detecting hybridization, wherein the oligonucleotide comprises a sequence of RCE1, preferably a unique sequence of, and determining the nucleotide sequence of the target nucleic acid to which the oligonucleotide is hybridized. The sequence can be determined according to various methods, including isolating the target nucleic acid, or a cDNA thereof and determining its sequence according to a desired method.

Oligonucleotides (nucleic acid) according to the present invention can be of any desired size, e.g., about 10-200 nucleotides, 12-100, preferably 12-50, 12-25, 14-16, at least about 15, at least about 20, etc. Such oligonucleotides can have non-naturally-occurring nucleotides, e.g., inosine. Such oligonucleotides have 100% identity or complementarity to a sequence of FIG. 1 or FIG. 2, or it can have mismatches or nucleotide substitutions, e.g., 1, 2, 3, 4, or 5 substitutions. In accordance with the present invention, the oligonucleotide can comprise a kit, where the kit includes a desired buffer (e.g., phosphate, tris, etc.), detection compositions, etc. The oligonucleotide can be labeled or unlabeled, with radioactive or non-radioactive labels as known in the art.

Anti-sense nucleic acid can also be prepared from a nucleic acid according to the present, preferably an anti-sense to a coding sequence of FIG. 1, 2, or 3. Antisense nucleic acid can be used in various ways, such as to regulate or modulate expression of RCE1, e.g., inhibit it, to detect its expression, or for in situ hybridization. These oligonucleotides can be used analogously to U.S. Pat. No. 5,576,208 describing inhibition of ras. For the purposes of regulating or modulating expression of RCE1, an anti-sense oligonucleotide can be operably linked to an expression control sequence.

The nucleic acid according to the present invention can be labelled according to any desired method. The nucleic acid can be labeled using radioactive tracers such as ³²P, ³⁵S, ¹²⁵I, ³H, or ¹⁴C, to mention only the most commonly used tracers. The radioactive labelling can be carried out according to any method such as, for example, terminal labeling at the 3′ or 5′ end using a radiolabeled nucleotide, polynucleotide kinase (with or without dephosphorylation with a phosphatase) or a ligase (depending on the end to be labelled). A non-radioactive labeling can also be used, combining a nucleic acid of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

A nucleic acid according to the present invention, including oligonucleotides, anti-sense nucleic acid, etc., can be used to detect expression of RCE1 in whole organs, tissues, cells, etc., by various techniques, including Northern blot, PCR, in situ hybridization, etc. Such nucleic acids can be particularly useful to detect disturbed expression, e.g., cell-specific and/or subcellular alterations, of RCE1. The levels of RCE1 can be determined alone or in combination with other genes products (oncogenes such as Ras), transcripts, etc.

A nucleic acid according to the present invention can be expressed in a variety of different systems, in vitro and in vivo, according to the desired purpose. For example, a nucleic acid can be inserted into an expression vector, introduced into a desired host, and cultured under conditions effective to achieve expression of a polypeptide coded for the nucleic acid. Effective conditions includes any culture conditions which are suitable for achieving production of the polypeptide by the host cell, including effective temperatures, pH, medias, additives to the media in which the host cell is cultured (e.g., additives which amplify or induce expression such as butyrate, or methotrexate if the coding nucleic acid is adjacent to a dhfr gene), cyclohexamide, cell densities, culture dishes, etc. A nucleic acid can be introduced into the cell by any effective method including, e.g., naked DNA, calcium phosphate precipitation, electroporation, injection, DEAE-Dextran mediated transfection, fusion with liposomes, associated with agents which enhance its uptake into cells, viral transfection. A cell into which a nucleic acid of the present invention has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells, e.g., COS-7, CHO, HeLa, LTK, NIH 3T3, yeast, insect cells, such as Sf9 (S. frugipeda), High Five Cells (Invitrogen), Drosophila, bacteria, such as E. coli, Streptococcus, bacillus, yeast, fungal cells, plants, embryonic stem cells (e.g., mammalian, such as mouse or human), cancer or tumor cells. Sf9 are preferred for insect expression; expression can be accomplished according to, e.g., O'Reilly et al., Baculovirus Expression Vectors: A Laboratory Manual, Freeman, N.Y., 1992. HEK293 mammalian cells can be used for mammalian overexpression. See, e.g., Collins et al., J Biol. Chem., 271:17349-17353 (1996). Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive expression, include, e.g., the endogenous promoter, MMTV, SV40; trp, lac, tac, or T7 promoters for bacterial hosts; or alpha factor, alcohol oxidase, or PGH promoters for yeast.

Another gene of interest can be introduced into the same host for purposes of, e.g., modulating expression RCE1, elucidating RCE1 function or that of the gene of interest. Genes of interest include other oncogenes, genes involved in the cell cycle, etc. Such genes can be the normal gene, or a variation, e.g., a mutation, chimera, polymorphism, etc.

A nucleic acid or polypeptide of the present invention can be used as a size marker in nucleic acid or protein electrophoresis, chromatography, etc. Defined restriction fragments can be determined by scanning the sequence for restriction sites, calculating the size, and performing the corresponding restriction digest. The RCE1 polypeptide can also be used as a 35.8 kd molecular weight marker for a protein gel. The RCE1 DNA disclosed herein can also be used as a 1472 bp marker on a DNA gel.

Another aspect of the present invention relates to the regulation of biological pathways in which a RCE1 gene is involved, particularly pathological conditions. For example: cell proliferation (e.g., cancer), growth control, morphogenesis, , 268:233-239, 1995; Bussey, Science, 272:225-226, 1996. For example, RCE1 is involved in the ras-dependent signal-transduction cascade. It is responsible for COOH-terminal processing of ras, a step in ras maturation. Over-expression of ras (wild-type, mutated, constitutive, etc., ras) leads to oncogenic activity. One approach to treating ras over-expression is inhibiting the ras maturation pathway so incompletely processed and inactive ras accumulates, eliminating or reducing its oncogenic effect. In accordance with the present invention, the ras maturation pathway can be inhibited by blocking RCE1 activity. Such blocking can be accomplished in various ways, including by administering RCE1 antibodies or other ligands, RCE1 peptides (especially those that bind to the CAAX motif but lack endoproteolytic activity), inhibitors of RCE1 endoprotease, anti-sense or double-stranded RNA (e.g., Fire et al., Nature, 391:806-811, 1998). Blocking agents can be identified according to the methods described herein or those available in the art.

One aspect of the invention relates to identifying compounds which modulate RCE1 activity. The activity can be modulated by increasing, reducing, antagonizing, promoting, stabilizing, etc. RCE1. In one method of the invention, RCE1 activity can be measured by reacting, in the presence of a test compound, a substrate comprising a CAAX polypeptide motif and a mammalian RCE1, under conditions effective for the mammalian RCE1 to proteolytically remove the AAX amino acid residues from the substrate and expose the substrate's Cys-COOH terminus; detecting the proteolytic removal of the AAX residues; and identifying whether the test compound modulates RCE1 activity by comparing the amount of proteolytic removal of the AAX residues in the presence and absence of the test compound.

A substrate that can be enzymatically digested, i.e., proteolytically removed, by RCE1 preferably comprises a CAAX recognition site, where an RCE1 cleaves between the cysteine and aliphatic amino acid residues, prenylated CAAX containing peptides, such as a farnsylated, or geranylgeranylated CAAX peptides. Any substrate is suitable if it can be acted upon by RCE1. Thus, a substrate can comprise other atoms, such as additional amino acid residues linked by peptide or other bonds, and can be modified in any desirable way. For example, a substrate can be affixed to a solid suport, e.g., comprising, latex, sepharose, silica, agarose, sephadex, cellulose, polysaccharides, glass, polymers, etc. A substrate can also be detectably labeled, e.g., with antibody, avidin, biotin, radioactive labels, aptamers, fluorescent labels, nucleic acid, etc. The substrate can also comprise phosphates, methyl groups, sugars, or lipids. In a preferred embodiment, the substrate contains a lipid, e.g., a cholesterol intermediate, such as a 15-carbon farnesyl or 20-carbon geranylgeranyl group. Preferably, the substrate is prenylated. In a preferred embodiment, the substrate is biotin-Lys-Lys-Ser-Lys-Thr-Lys-(Farnesyl)Cys-Val-Ile-Met, more generally it is a geranylgeranylated CAAX containing peptide. The test compound is preferably reacted with an RCE1 in a milieu in which RCE1 cleaves the substrate. Such a milieu can be referred to as effective conditions. These conditions can be determined in the absence of the test compound to establish a baseline activity, e.g., as in a control. The effective reaction conditions can be routinely selected, e.g., using salts, buffers, reducing and/or oxidizing agents, pH's, etc. When utilizing a substrate comprising a CAAX motif, effective cleavage results in the removal of the AAX residues from the substrate, exposing the Cys-COOH terminus.

After the step of reacting the substrate, test compound, and RCE1, under conditions in which proteolysis can be achieved, the next step is to determine whether proteolysis occurred. Detecting proteolysis, like the selection of effective reaction conditions, can be optimized in the absence of the test compound to establish a baseline activity for RCE1. Generally, proteolysis detection involves identifying a product of the reaction. For example, when the cleavage site is an amino acid sequence, complete proteolysis of the substrate results in cleavage products having novel 3′ and 5′ termini. The products can be detected directly, e.g., by chromatography, electrophoresis, mass spectroscopy, immunoassay etc., or the termini can be detected, e.g., by measuring the appearance or a property of the novel termini. In a preferred embodiment, where the substrate comprises CAAX and cleavage results in the appearance of the Cys-COOH termini, the latter is detected by methylating it using a methylase and a labeled-methionine-substrate. In a more preferred aspect, the methylase is a prenyl protein-specific methyltransferase (PPSMT) and the methionine-substrate is ³H-S-adenosyl methionine. The resultant labeled RCE1 substrate can be separated from free label conventionally. For example, if the RCE1 substrate is labeled at its 5′ end with biotin, it can be captured by avidin which is preferably attached to beads. In addition, the RCE1 substrate can be attached to a solid surface, a magnetic bead, etc. and processed conventionally.

A methylase can be purified, enriched, provided as a component of a cell extract, e.g., from a mammalian cell or yeast cell, etc. The extract or lysate can be obtained from various cells, including cells transformed with a methylase gene, e,g., yeast STE14. See, e.g., Hrycyna et al., Methods in Enzymology, 250:251-266, 1995.

The RCE1 component (i.e., a polypeptide or endoproteolytic fragment thereof) can be added to the reaction mixture in a variety of forms, e.g., substantially purified, as a component of cell membranes (such as, endoplasmic reticulum), or as a soluble extract. In each case, the RCE1 polypeptide can be obtained from a natural source, a recombinant source, or it can be produced synthetically (produced chemically or enzymatically, e.g., cleavage of a full-length RCE1 ).

Preferably, the RCE1 is expressed in a cell line transformed with an RCE1 coding sequence (e.g., a cDNA, a gene, a genomic fragment, etc.). In the latter case, the RCE1 is present as a heterologous component of the cell; by heterologous, it is meant that the RCE1 is not only expressed in a cell line of a different species, but it is also coded for by a coding sequence that has been introduced into the cell, e.g., by transfection, transformation, etc. Preferably, the RCE1 is expressed at high levels in the cell. A human RCE1, or a fragment thereof, is a preferred coding sequence. See, e.g., FIG. 1. A useful fragment of RCE1 comprises an endoprotease activity and substrate binding activity, e.g. amino acids 19-329.

In a preferred aspect of the invention, the RCE1 is provided as a cell lysate, e.g., cells transformed with RCE1 are lysed and the resulting lysate is used directly in the assay, i.e., a crude lysate. The crude lysate comprising the recombinant RCE1 can optionally be refined or enriched for RCE1. For instance, e.g., a membrane fraction can be isolated, etc. For example, cells expressing RCE1 (such as HEK293) are harvested, washed in PBS+20 mM EDTA, lysed by douncing in hypotonic lysis buffer or by using nitrogen cavitation, subjected to a low speed spin to remove insoluble material and cell debris (including unbroken cells and nuclei), and then centrifuged at 100,000 g for an amount of time effective to pellet membranes.

A purpose of the assay is to select and identify compounds which modulate RCE1 activity. Thus, proteolysis detection is typically performed in the presence and absence of the test compound. Whether a compound modulates RCE1 activity can be determined routinely, e.g., by determining whether more or less proteolysis has occurred in the presence of the test compound.

The assay can also be conducted in whole cells. For example, cells overexpressing an RCE1 have a transformation promoting activity. Over-expression can be achieved in a cell by genetic engineering means, e.g., transforming an RCE1 gene operably linked to a robust promoter, by selecting cell lines (such as HEK293) for such activity, etc. Agents can be administered to such cells and tested for their ability to inhibit transformation, e.g., by monitoring cell morphology, etc. See, e.g., U.S. Pat. No. 5,688,655. Assays can also be carried out as described in U.S. Pat. Nos. 5,710,171; 5,703,241; 5,585,359; 5,557,729; 5,532,359; 5,470,832; 5,420, 245; 5,185,248.

Compounds identified in this or other manners can be useful to modulate RCE1 activity in a cell, a tissue, a whole organism, in situ, in vitro (test tube, a solid support, etc.), in vivo, or in any desired environment. In general, a compound having such an in vitro activity will be useful in vivo to modulate a biological pathway associated with RCE1, e.g., to treat a pathological condition associated with the biological and cellular activities mentioned above. The present invention thus also relates to the treatment and prevention of diseases and pathological conditions associated with ras-mediated signal transduction, e.g., cancer, diseases associated with abnormal cell proliferation. For example, the invention relates to a method of treating cancer comprising administering, to a subject in need of treatment, an amount of a compound effective to treat the disease, where the compound is a regulator of RCE1 gene or polypeptide expression. Treating the disease can mean, delaying its onset, delaying the progression of the disease, improving or delaying clinical and pathological signs of disease. A regulator compound, or mixture of compounds, can be synthetic, naturally-occurring, or a combination. A regulator compound can comprise amino acids, nucleotides, hydrocarbons, lipids, polysaccharides, etc. A regulator compound is preferably a regulator of RCE1, e.g., inhibiting or increasing its mRNA, protein expression, or processing. Expression can be regulated using different agents, e.g., an anti-sense nucleic acid, a ribozyme, an aptamer, a synthetic compound, or a naturally-occurring compound. To treat the disease, the compound, or mixture, can be formulated into pharmaceutical composition comprising a pharmaceutically acceptable carrier and other excipients as apparent to the skilled worker. See, e.g., Remington's Pharmaceutical Sciences, Eighteenth Edition, Mack Publishing Company, 1990. Such composition can additionally contain effective amounts of other compounds, especially for treatment of cancer.

The present invention also relates to antibodies which specifically recognize a RCE1 polypeptide. Antibodies, e.g., polyclonal, monoclonal, recombinant, chimeric, can be prepared according to any desired method. For example, for the production of monoclonal antibodies, a polypeptide according to FIG. 1, can be administered to mice, goats, or rabbit subcutaneously and/or intraperitoneally, with or without adjuvant, in an amount effective to elicit an immune response. The antibodies can also be single chain or FAb. The antibodies can be IgG, subtypes, IgG2a, IgG1, etc. Antibodies can also be generated by administering naked DNA See, e.g., U.S. Pat. Nos. 5,703,055; 5,589,466; 5,580,859.

An antibody specific for RCE1 means that the antibody recognizes a defined sequence of amino acids within or including the RCE1 amino acid sequence of FIG. 1 or FIG. 3. Thus, a specific antibody will bind with higher affinity to an amino acid sequence, i.e., an epitope, found in FIG. 1 or 3 than to epitope(s) found in a different protein, e.g., as detected and/or measured by an immunoblot assay. Thus, an antibody which is specific for an epitope of RCE1 is useful to detect the presence of the epitope in a sample, e.g., a sample of tissue containing RCE1 gene product, distinguishing it from samples in which the epitope is absent. Such antibodies are useful as described in Santa Cruz Biotechnology, Inc., Research Product Catalog, and can be formulated accordingly, e.g., 100 μg/ml. A specific antibody has been raised to the carboxy terminal 12 residues of human RCE1: Glu-Arg-Ala-Gly-Asp-Ser-Glu-Ala-Pro-LeuCys-Ser.

In addition, ligands which bind to an RCE1 polypeptide according to One present invention, or a derivative thereof, can also be prepared, e.g., using synthetic peptide libraries or aptamers (e.g., Pitrung et al., U.S. Pat. No. 5,143,854; Geysen et al., 1987, J. Immunol. Methods, 102:259-274; Scott et al., 1990, Science, 249:386; Blackwell et al., 1990,, Science, 250:1104; Tuerk et al., 1990, Science, 249: 505.

Antibodies and other ligands which bind RCE1 can be used in various ways, including as therapeutic, diagnostic, and commercial research tools, e.g., to quantitate the levels of RCE1 polypeptide in animals, tissues, cells, etc., to identify the cellular localization and/or distribution of RCE1, to purify RCE1, or a polypeptide comprising a part of RCE1, to modulate the function of RCE1, etc. Antibodies to RCE1, or a derivative thereof, can be used in Western blots, ELIZA, immunoprecipitation, RIA, etc. The present invention relates to such assays, compositions and kits for performing them, etc. Similarly, antibodies that bind RCE1 can be used to immunoprecipitate RCE1 from cell lysates to identify substances that bind RCE1.

An antibody according to the present invention can be used to detect RCE1 polypeptide or fragments thereof in various samples, including tissue, cells, body fluid, blood, urine, cerebrospinal fluid. A method of the present invention comprises contacting a ligand which binds to a peptide of FIG. 1 or 3 under conditions effective, as known in the art, to achieve binding, detecting specific binding between the ligand and peptide. By specific binding, it is meant that the ligand attaches to a defined sequence of amino acids, e.g., within or including the amino acid sequence of FIG. 1 or FIG. 3. The antibodies or derivatives thereof can also be used to inhibit expression of RCE1 or a fragment thereof. The levels of RCE1 polypeptide can be determined alone or in combination with other gene products. In particular, the amount (e.g., its expression level) of RCE1 polypeptide can be compared (e.g., as a ratio) to the amounts of other polypeptides in the same or different sample, e.g., ras, Ftase, etc. A ligand for RCE1 can be used in combination with other antibodies, e.g., antibodies that recognize oncological markers of cancer, including, ras, etc. In general, reagents which are specific for RCE1 can be used in diagnostic and/or forensic studies according to any desired method, e.g., as U.S. Pat. Nos. 5,397,712; 5,434,050; 5,429,947.

The present invention also relates to a labelled RCE1 polypeptide, prepared according to a desired method, e.g., as disclosed in U.S. Pat. No. 5,434,050. A labelled polypeptide can be used, e.g., in binding assays, such as to identify substances that bind or attach to RCE1, to track the movement of RCE1 in a cell, in an in vitro, in vivo, or in situ system, etc. Similarly, an antibody that binds to RCE1 can be used to immunoprecipitate RCE1 from a cell lysate to identify substances which can co-precipitate with RCE1.

A nucleic acid, polypeptide, antibody, RCE1 ligand etc., according to the present invention can be isolated. The term “isolated” means that the material is in a form in which it is not found in its original environment, e.g., more concentrated, more purified, separated from component, etc. An isolated nucleic acid includes, e.g., a nucleic acid having the sequence of RCE1 separated from the chromosomal DNA found in a living animal. This nucleic acid can be part of a vector or inserted into a chromosome (by specific gene-targeting or by random integration at a position other than its normal position) and still be isolated in that it is not in a form which it is found in its natural environment. A nucleic acid or polypeptide of the present invention can also be substantially purified. By substantially purified, it is meant that nucleic acid or polypeptide is separated and is essentially free from other nucleic acids or polypeptides, i.e., the nucleic acid or polypeptide is the primary and active constituent.

The present invention also relates to a transgenic animal, e.g., a non-human-mammal, such as a mouse, comprising a RCE1 nucleic acid. Transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology. See, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384 (1980); Palmiter et al., Cell, 41:343-345 (1985); Palmiter et al., Ann. Rev. Genet., 20:465-499 (1986); Askew et al., Mol Cell. Bio., 13:4115-4124, 1993; Games et al. Nature, 373:523-527, 1995; Valancius and Smithies, Mol. Cell Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993. A nucleic acid according to the present invention can be introduced into any non-human mammal, including a mouse (Hogan et al., 1986, in Manipulating the Mouse Embryo: A Laboratory Manual, Cold Spring Harbor Laboratory, Cold Spring Harbor, N.Y.), pig (Hammer et al., Nature, 315:343345, 1985), sheep (Hammer et al., Nature, 315:343-345, 1985), cattle, rat, or primate. See also, e.g., Church, 1987, Trends in Biotech. 5:13-19; Clark et al., 1987, Trends in Biotech. 5:20-24; and DePamphilis et al., 1988, BioTechniques, 6:662-680. In addition, e.g., custom transgenic rat and mouse production is commercially available. These transgenic animals are useful as a cancer model, e.g., to test drugs, or as food for a snake.

Generally, the nucleic acids, polypeptides, antibodies, etc. of the present invention can be prepared and used as described in, U.S. Pat. Nos. 5,501,969, 5,506,133, 5,441,870; WO 90/00607; WO 91/15582;

For other aspects of the nucleic acids, polypeptides, antibodies, etc., reference is made to standard textbooks of molecular biology, protein science, and immunology. See, e.g., Davis et al. (1986), Basic Methods in Molecular Biology, Elsevir Sciences Publishing, Inc., New York; Hames et al. (1985), Nucleic Acid Hybridotion, IL Press, Molecular Cloning, Sambrook et al.; Current Protocols in Molecular Biology, Edited by F. M. Ausubel et al., John Wiley & Sons, Inc; Current Protocols in Human Genetics, Edited by Nicholas C. Dracopoli et al., John Wiley & Sons, Inc.; Current Protocols in Protein Science; Edited by John E. Coligan et al., John Wiley & Sons, Inc.; Current Protocols in Immunology; Edited by John E. Coligan et al., John Wiley & Sons, Inc.

### EXAMPLE

An assay to demonstrate RCE1 can be a coupled assay linked to the prenyl-directed carboxymethylase (yeast homolog: STE14; See, e.g., Methods Enzymol 1995; 250:251-66). A biotinylated, prenylated peptide substrate (e.g., Biotin-LysLys-Ser-Lys-Thr-Lys-(Farnesyl)Cys-Val-Ile-Met was based on the C-terminal sequence of K-Ras-4B). In short, the human RCE1 expressing insect cell membranes cleave the last three amino acids to expose the (Farnesyl)Cys-carboxyl group; subsequently, endogenous (or exogenous) prenyl-cysteine directed carboxymethylase would methylate the exposed carboxyl group using the co-substrate ³H-S-adenosyl methionine. The resulting label is incorporated into the substrate peptide is quantified using streptavidin-coated SPA beads.

Standard assay is performed in 96-well sample plates (Wallac Part No. 1450-401) with a total assay volume of 100 μl which generally contains: 50 μl compound, 25 μl membranes and 25 μl ³H-SAM/substrate added in that order. Final concentration of HEPES pH 7.4 is 100 mM.

A volume of 25 μl of membranes in 100 mM HEPES pH 7.4 is added to each well, followed by 25 μl diluted substrate (protease substrate Biotin-Lys-Lys-Ser-Lys-Thr-Lys-(Farnesyl)Cys-Val-Ile-Met is stored at −20° C. in 100% DMSO but is diluted in 10% DMSO to the required working concentration immediately before use). To this is added the label i.e. ³H-SAM (˜85Ci.mmol; 1mCi/ml; 12 μM), typically 0.2 μl per well made up to 25 μl with 100 mM HEPES pH 7.4. The plate is then sealed and incubated at room temperature for 60 mins. This reaction is stopped by adding 150 μl Stop Mix which contains SPA beads (250 μg) in PBS pH 7.1+5 mM EDTA+0.1% Tween-20. The plate is sealed again and the beads are left to settle overnight before reading.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated by reference.

From the foregoing description, one skilled in the art can easily ascertain the essential characteristics of this invention, and without departing from the spirit and scope thereof, can make various changes and modifications of the invention to adapt it to various usages and conditions.

What is claimed:

1. An isolated mammalian RCE1 polypeptide, or a biologically-active polypeptide fragment thereof, with the proviso that said polypeptide is not a polypeptide coded for by AA021859, AA072190, AA154658, AA154864, AA168614, AA218396, AA619282, AA790517, C77052, C86966, W14344, or W57162.

2. An isolated mammalian RCE1, or a biologically-active polypeptide fragment thereof, of claim 1, wherein said polypeptide has an endonuclease activity, a substrate binding activity, or an immunogenic activity.

3. An isolated mammalian RCE1, or a biologically-active polypeptide fragment thereof, of claim 1, wherein the substrate binding activity is binding to a prenylated CAAX peptide substrate.

4. An isolated mammalian RCE1, or a biologically-active polypeptide fragment thereof, of claim 1 which is human.

5. An isolated mammalian RCE1, or a biologically active polypeptide fragment thereof, of claim 1 which is mouse.

6. An isolated mammalian RCE1 of claim 1 which is human, and comprises amino acid 1 to amino acid 329 as set forth in FIG. 1.

7. An isolated mammalian RCE1 of claim 1, which is human, and comprises contiguously amino acid 1 to amino acid 230 and amino acid 252 to amino acid 329 as set forth in FIG. 1.

8. An isolated mammalian RCE1 of claim 1, comprising amino acids 1 329 as set forth in FIG. 3.

9. An isolated RCE1 of claim 1, coded for by a naturally obtainable nucleic acid which hybridizes under stringent conditions to the DNA sequence set forth in FIG. 1, or its complement, with the proviso that the sequence is not AA021859, AA072190, AA154658, AA154864, AA168614, AA218396, AA619282, AA790517, C77052, C86966, W14344, W57162, yeast RCE1, or a fragment of yeast RCE1.

10. An isolated RCE1 of claim 9, comprising at least about 95% amino acid identity to the amino acid sequence set forth in FIG. 1.

11. An isolated RCE1 of claim 1, coded for by a naturally obtainable nucleic acid which hybridizes under stringent conditions to the DNA sequence set forth in FIG. 2, or its complement, with the proviso that sequence is not a AA021859, AA072190, AA154658, AA154864, AA168614, AA218396, AA619282, AA790517, C77052, C86966, W14344, W57162, yeast RCE1, or a fragment of yeast RCE1.

12. An isolated nucleic acid comprising a nucleotide sequence coding for a mammalian RCE1 polypeptide, or a biologically-active polypeptide fragment thereof, with the proviso that sequence is not AA021859, AA072190, AA154658, AA154864, AA168614, AA218396, AA619282, AA790517, C77052, C86966, W14344, or W57162.

13. An isolated nucleic acid of claim 12, wherein said coded for polypeptide has a has an endonuclease activity, a substrate binding activity, or an immunogenic activity.

14. An isolated nucleic acid of claim 13, wherein the substrate binding activity is binding to a prenylated CAAX peptide substrate.

15. An isolated nucleic acid of claim 12 which is human.

16. An isolated nucleic acid of claim 12, wherein the nucleic acid sequence codes for amino acid 1 to amino acid 329 as set forth in FIG. 1.

17. An isolated nucleic acid of claim 12, wherein the nucleic acid codes contiguously for 1 to amino acid 230 and amino acid 252 to amino acid 329 as set forth in FIG. 1.

18. An isolated nucleic acid of claim 12, having the complete coding nucleotide sequence set forth in FIG. 1, or a complement thereto.

19. An isolated nucleic acid of claim 18, except where one or more amino acid positions are substituted or deleted, or both, and the polypeptide coded for by the nucleic acid is biologically-active.

20. An isolated nucleic acid of claim 18, wherein the one or more substituted amino acid positions are substituted by homologous amino acids.

21. An isolated nucleic acid of claim 12, wherein the nucleic acid sequence codes for an amino acid sequence selected from FIG. 1, and said amino acid sequence has an endonuclease activity, a substrate binding activity, or an immunogenic activity.

22. An isolated nucleic acid of claim 12, coded for by a naturally obtainable nucleic acid sequence which hybridizes under stringent conditions to the DNA sequence set forth in FIG. 1, or a complement thereto, with the proviso that the nucleic acid is not AA021859, AA072190, AA154658, AA154864, AA168614, AA218396, AA619282, AA790517, C77052, C86966, W14344, W57162, yeast RCE1, or a fragment of yeast RCE1.

23. An isolated nucleic acid of claim 12, consisting essentially of any continuous sequence of 12-100 base pairs, or a complement thereto, selected from the nucleotide sequence set forth in FIG. 1.

24. An isolated nucleic acid of claim 23, at least one but not more than five, nucleotide substitutions from said sequence.

25. An isolated nucleic acid of claim 23, further comprising a detectable label.

26. An isolated nucleic acid of claim 12, having the complete coding nucleotide sequence set forth in FIG. 2, or a complement thereto.

27. An isolated nucleic acid of claim 12, wherein the nucleotide sequence is operably linked to an expression control sequence.

28. An isolated nucleic acid of claim 12, wherein the nucleic acid comprises a nucleotide sequence which is naturally-obtainable.

29. An isolated nucleic acid of claim 12, wherein the nucleic acid codes for said polypeptide without interruption.

30. An isolated nucleic acid of claim 12, wherein the nucleic acid is DNA or RNA.

31. An isolated nucleic acid of claim 11, wherein the coded for biologically-active polypeptide has an endonuclease activity, a substrate binding activity, or an immunogenic activity.

32. A method of expressing in transformed host cells, a mammalian RCE1 polypeptide coded for by a nucleic acid, comprising:
culturing transformed host cells containing a nucleic acid according to claim 12 under conditions effective to express the polypeptide.

33. A method of claim 32, further comprising isolating the polypeptide.

34. A method of claim 32, further comprising modulating expression of the polypeptide.

35. An isolated polypeptide produced by a method of claim 32.

36. A transformed host cell containing a nucleic acid of claim 12.

37. A vector comprising a nucleic acid of claim 12.

38. A vector comprising a nucleic acid of claim 12.

39. A transgenic non-human mammal comprising a nucleic acid of claim 12.

40. A method of identifying compounds that modulate mammalian RCE1 activity comprising:
reacting, in the presence of a test compound, a substrate comprising a terminal CAAX polypeptide motif and a mammalian RCE1, or endoproteolytic fragment thereof, under conditions effective for the mammalian RCE1, or said fragment, to proteolytically remove the AAX amino acid residues from the substrate and expose the substrate's Cys-COOH terminus;
detecting the proteolytic removing of the AAX residues; and
identifying whether the test compound modulates RCE1 endoproteolytic activity by comparing the amount of proteolytic removing of the AAX residues in the presence and absence of the test compound.

41. A method of claim 40, wherein the substrate is prenylated.

42. A method of claim 40, wherein the substrate is biotin-Lys-Lys-Ser-Lys-Thr-Lys-(Farnesyl)Cys-Val-Ile-Met.

43. A method of claim 40, wherein the detecting the proteolytic removing is accomplished by:
detecting the Cys-COOH terminus of the substrate exposed by the proteolysis by the mammalian RCE1.

44. A method of claims 40, wherein the detecting the proteolytic removing is accomplished by:
methylating the Cys-COOH terminus of the substrate exposed by the proteolysis by the mammalian RCE1 using detectably-labeled-S-adenosyl methionine;
detecting the detectably-labeled methylated Cys-COOH.

45. A method of claim 40, wherein the detecting the proteolytic removing is accomplished by:
methylating the Cys-COOH terminus of the substrate exposed by the proteolysis of the mammalian RCE1 polypeptide using detectably-labeled-S-adenosyl methionine, whereby the methylating is performed by a methyltransferase and results in a detectably-labeled and methylated Cys-COOH terminus.

46. A method of claim 40, wherein the methyltransferase is prenyl protein specific.

47. A method of claim 40, wherein the mammalian RCE1 is substantially purified.

48. A method of claim 40, wherein the mammalian RCE1 is present as a heterologous component of cell membranes.

49. A method of claim 40, wherein the mammalian RCE1 is present as a heterologous component of a cell membrane extract.

50. A method of claim 40, wherein the polypeptide substrate is biotin-Lys-Lys-Ser-Lys-Thr-Lys-(Farnesyl)Cys-Val-Ile-Met and detecting the proteolytic removing is accomplished by:
methylating the Cys-COOH terminus of the substrate exposed by the proteolysis of the mammalian RCE1 polypeptide using detectably-labeled S-adenosyl methionine, whereby the methylating is performed by a methyltransferase and results in a detectably-labeled and methylated Cys-COOH terminus of the substrate;
capturing the substrate using strepavidin-coated beads;
quantifying the detectable label present in the captured substrate.

51. A method of claim 40, wherein the mammalian RCE1 is human or mouse.

52. A method of modulating a ras-dependent signal transduction pathway comprising, introducing a nucleic acid of claim 12, or its anti-sense, into said cell under conditions whereby said nucleic acid is expressed in an effective amount to modulate said signal transduction pathway.

53. A method of claim 52, wherein said RCE1 is human.

54. An isolated antibody which is specific for a RCE1.

55. An isolated antibody of claim 52, which binds to an amino acid sequence of amino acid 1 to amino acid 311 as set forth in FIG. 1.

56. An isolated antibody of claim 52 which is specific for Glu-Arg-Ala-Gly-Asp-Ser-Glu-Ala-Pro-Leu-Cys-Ser
